(19) United States
(12) Patent Application Publication
(10) Pub. No.: US 2023/0404991 A1
(43) Pub. Date: Dec. 21, 2023

(54) FIBROUS PELLET FOR ORAL APPLICATIONS AND METHODS THEREOF

(71) Applicant: Barnhardt Manufacturing Co., Charlotte, NC (US)

(72) Inventor: Wade Monroe Hubbard, JR., Charlotte, NC (US)

(21) Appl. No.: 18/335,575

(22) Filed: Jun. 15, 2023

Related U.S. Application Data
(60) Provisional application No. 63/352,680, filed on Jun. 16, 2022.

(51) Int. Cl. A61K 31/465; A61J 3/06; A61K 31/265; A61K 31/00; A61K 9/70; A24B 15/16; D04H 1/425; D04H 1/435; D04H 1/4291; D04H 1/54; D04H 1/492; D04H 1/74; D04H 1/4382

(52) U.S. Cl. CPC A61K 31/465; A61J 3/06; A61K 31/265; A61K 31/658; A61K 9/7007; A24B 15/16; D04H 1/425; D04H 1/435; D04H 1/4291; D04H 1/54; D04H 1/492; D04H 1/74; D04H 1/43825; D10B 2509/00

(57) ABSTRACT

An improved fibrous pellet for oral applications is disclosed herein. The fibrous pellet has improved physical and mechanical properties due to an improved manufacturing method that produces a more uniform and structurally sound fibrous pellet compared with the prior art. The fibrous pellet described herein may be configured for intra-oral delivery of active substances as well as the intra-oral absorption of fluids.

### FIELD

The present disclosure relates generally to a fibrous pellet used for the absorption of fluids or the release of fluids in the oral cavity.

### BACKGROUND OF THE INVENTION

Fibrous pellets can be used in various oral applications such as delivering active ingredients, i.e. as nicotine in the case of nicotine pouches or snus, as well as in the medical field for absorbing fluids in the oral cavity. Traditionally, pellets for oral applications are in a “rolled” or “spun” form. These traditional pellets may easily become unraveled and leave fibers behind in the oral cavity. Another issue with the traditional pellets is the inability to properly manage fluid intake and release. The manufacturing process can result in a drastic size and weight variability amongst pellets. Further, the process is not scalable and provides no opportunity to improve the mechanical properties of the pellet.

Therefore, there is a need for an improved fibrous pellet product for oral applications and an improved method of manufacturing said fibrous pellet product.

### BRIEF SUMMARY

In view of the above, it is an object of the present disclosure to provide a fibrous pellet with improved and tunable properties, as well as an improved method of manufacturing said fibrous pellets to tune the mechanical and fluid management properties.

In one aspect, a fibrous pellet is disclosed. The fibrous pellet comprising a plurality of fibers oriented linearly relative to the X axis, Y axis, and Z axis. In some embodiments, the plurality of fibers comprise polyester. Additionally, or alternatively, the plurality of fibers comprise a bi-component fiber. In some embodiments, there are a mixture of fiber types. These fiber types can include cotton, rayon, jute, hemp, flax, polyester, bi-component, polypropylene, and any combination. In some embodiments, combinations of bondable fibers with cellulosic fibers are desired. In further embodiments, fibers of various shape can be used such as circular, tri-lobal, triangular, polygonal, flat, oval, lobular, dog bond, square, I beam, and star.

In some embodiments, the fibrous pellet has a dynamic heterogeneous morphology. Additionally, or alternatively, the fibrous pellet has a dynamic heterogeneous porosity and permeability. In some embodiments, the fibrous pellet is configured for time-delayed release of an active ingredient.

In another aspect, a method of manufacturing a fibrous pellet is disclosed. The method comprising: carding a fiber, cross-lapping the fibers to create a layered substrate, bonding the layered substrate, and die-cutting to form pellets. In some embodiments, the method comprises applying an active ingredient to the fiber, the aligned fiber, the layered substrate, the bonded substrate, or the pellets. In some embodiments, bonding the layered substrate comprises needle-punching, hydro-entangling, thermal bonding, ultrasonic bonding, or combinations thereof. In some embodiments, the method further includes applying an active ingredient to the fiber, the aligned fiber, the layered substrate, the bonded substrate, or the pellets.

In another aspect, a fibrous pellet formed by the process of: carding a fiber to generate aligned fibers, cross-lapping the aligned fibers to create a layered substrate, bonding the layered substrate to create bonded substrate, and die-cutting the bonded substrate into pellets is disclosed. In some embodiments, bonding the layered substrate comprises needle-punching, hydro-entangling, thermal bonding, ultrasonic bonding, or combinations thereof. In some embodiments, the process further includes applying an active ingredient to the fiber, the aligned fiber, the layered substrate, the bonded substrate, or the pellets.

Additional features, aspects, and advantages of the invention will be set forth in the detailed description which follows, and in part will be readily apparent to those skilled in the art from that description or recognized by practicing the invention as described herein.

### BRIEF DESCRIPTION OF THE DRAWINGS

The drawings described herein are for illustrative purposes for selected embodiments and not all possible implementations, and are not intended to limit the scope of the present disclosure.

FIG. 1 illustrates a process for manufacturing a fibrous pellet, in accordance with the prior art.

FIG. 2 illustrates a process for manufacturing a fibrous pellet, in accordance with an embodiment of the present invention.

FIG. 3 illustrates an exemplary work-flow set up for substrate-making, in accordance with an embodiment of the present invention.

FIGS. 4A and 4B illustrate an apparatus for cross-lapping, in accordance with an embodiment of the present invention.

FIGS. 5A and 5B illustrates substrate bonding in accordance with an embodiment of the present invention.

FIGS. 6A and 6B illustrates needle-punching in accordance with an embodiment of the present invention.

FIG. 7 illustrates hydro-entangling in accordance with an embodiment of the present invention.

FIGS. 8A and 8B illustrate the mechanical properties of the improved fibrous pellet, in accordance with an embodiment of the present invention, and the prior art fibrous pellet.

FIG. 9 illustrates a comparative top view and perspective view of the improved fibrous pellet, in accordance with an embodiment of the present invention and the prior art fibrous pellet.

FIG. 10 illustrates a process for evaluating compression and recovery properties of fibrous pellets.

FIGS. 11A and 11B illustrate 1st and 3rd cycle compression and recovery tests for both the improved fibrous pellet, in accordance with an embodiment of the present invention, and the prior art pellet while wet.

FIGS. 12A and 12B illustrate 1st and 3rd cycle compression and recovery tests for both the improved fibrous pellet, in accordance with an embodiment of the present invention, and the prior art pellet while dry.

FIGS. 13A and 13B illustrate compression work of the improved fibrous pellet, in accordance with an embodiment of the present invention, and the prior art pellet.

FIGS. 14A and 14B illustrate thickness changes of the improved fibrous pellet, in accordance with an embodiment of the present invention, and the prior art pellet.

FIGS. 15A and 15B illustrate recovery work of the improved fibrous pellet, in accordance with an embodiment of the present invention, and the prior art pellet.

FIG. 16 represents the percent thickness recovery of the improved fibrous pellet, in accordance with an embodiment of the present invention, and the prior art pellet.

FIGS. 17A and 17B illustrate fluid loss in a squeeze-out test and free fluid loss test in both the improved fibrous pellet, in accordance with an embodiment of the present invention, and the prior art pellet.

FIGS. 18A and 18B illustrate percent fluid retention during squeeze out and free fluid loss tests in both the improved fibrous pellet, in accordance with an embodiment of the present invention, and the prior art pellet.

FIGS. 19A and 19B illustrate fluid retention capacity during squeeze out and free fluid loss tests in both the improved fibrous pellet, in accordance with an embodiment of the present invention, and the prior art pellet.

### DETAILED DESCRIPTION OF EMBODIMENTS OF THE INVENTION

The present invention will now be described more fully hereinafter with reference to the accompanying drawings in which exemplary embodiments of the invention are shown. However, the invention may be embodied in many different forms and should not be construed as limited to the representative embodiments set forth herein.

Measurements, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

The improved fibrous pellet, referred to herein as “pellet” and “fibrous pellet,” of the present disclosure is a pellet comprised of a plurality of fibers. The pellet may have a tunable shape such as a sphere, disc, cylinder, triangle, star, diamond, etc. The physical and mechanical properties of the pellet are tunable due to the unique method of manufacturing the pellets. Properties such as fiber orientation, compressive and shear properties, density, porosity, and permeability may be tuned to produce a pellet with desirable functionality and properties. The pellets may exhibit the ability change dynamically regarding porosity and permeability during manipulation by the end user. These new properties are possible through the manipulation of fiber orientation and through localized and discrete bonding. The improved fibrous pellets described herein may be used in various applications, including but not limited to buccal, sublingual, and intra-oral substance delivery. Additionally, or alternatively, the fibrous pellets may be used without an applied active ingredient to absorb oral fluids.

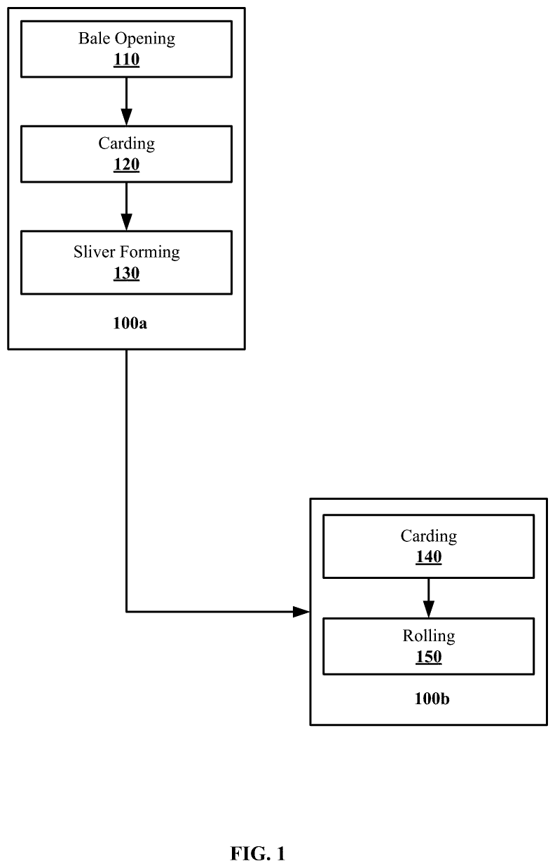

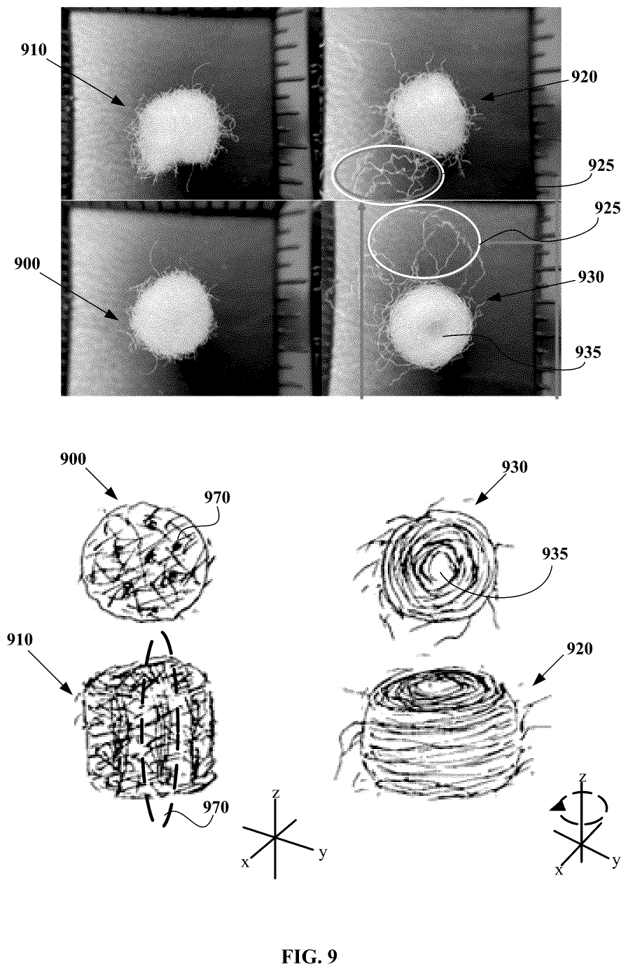

The prior art process for manufacturing fibrous pellets is illustrated in FIG. 1. The process is performed generally in two procedures; the first is sliver-making 100a, and the second is pellet-making 100b. Sliver-making 100a starts at block 110, a bale of fiber is opened. The fiber used in the prior art pellet is usually cotton or cellulose. The fiber is then carded at block 120. Carding includes separating the fiber tufts into individual fibers. After carding, the fiber strands are formed into slivers at block 130. Pellet-making 100b begins at block 140, where the slivers are carded, and at block 150 the fibers are rolled or spun into a pellet. Since these pellets are formed by rolling or spinning the fibers the fiber orientation within the pellet is only in the radial plane and only frictional forces between the fibers are holding the pellet shape. This typically results in the pellet unraveling and having extraneous fibers 925 as can be seen with the prior art pellets 930 in FIG. 9.

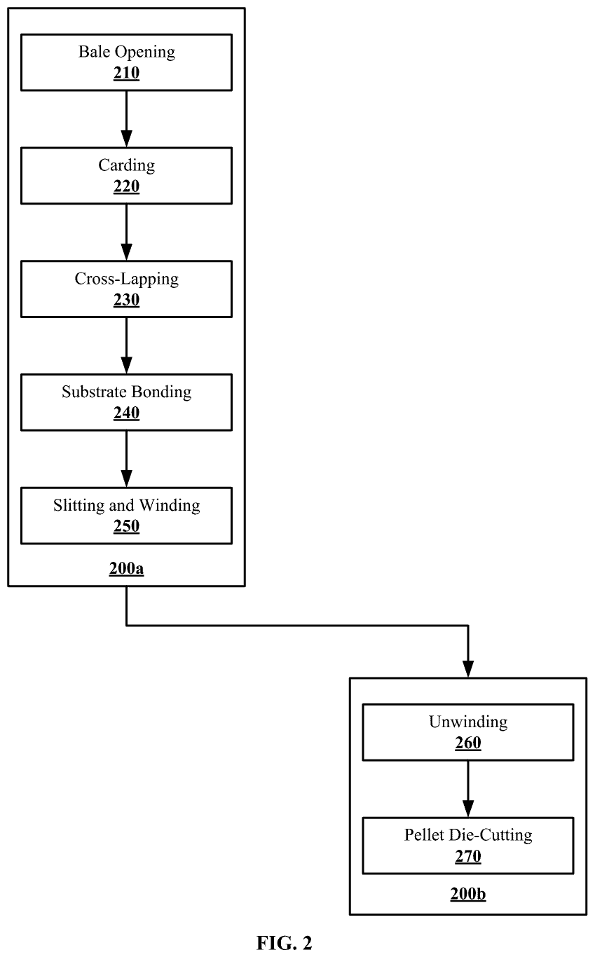

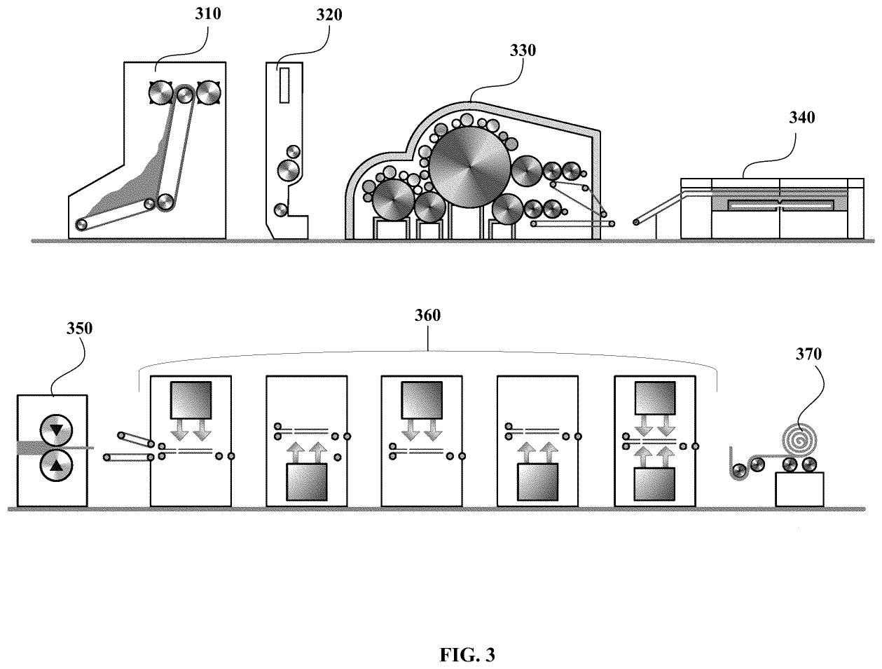

The improved method, disclosed herein, is illustrated in FIG. 2 and FIG. 3. The improved process may be performed in two procedures; the first procedure is substrate-making 200a, and the second procedure is pellet-forming 200b.

In some embodiments, the fiber used to form the fibrous pellet, or pellet, is polyester. The polyester fiber may have a denier of between 1 and 40 denier and more preferably between 1.5 and 20 denier. In some embodiments the polyester fiber may be solely composed of 6 denier fiber, while in other embodiments the polyester used will be a combination of multiple denier fibers as to create heterogeneity. The polyester fiber may comprise about 0.1% to about 1.0% finish. The polyester fiber may have approximately 1 to 20 crimps per inch and more preferably 7.5 crimps per inch to 12.5 crimps per inch. Additionally, the polyester fiber may be approximately to 2.5 inches in length. The polyester fiber may be used to make a polyester substrate that is between approximately 100 to 600 grams per square meter with a more preferred range between 200 to 500 grams per square meter. The polyester substrate may be used to make pellets that are approximately 3 to 15 mm in diameter and approximately 3 to 15 mm in height.

The substrate-making procedure 200a begins with opening a bale of fiber at block 210. The fibers are removed from the bale and gently separated into manageable tuft sizes to minimize fiber damage while separating. In some embodiments, the fiber tufts are added to an apparatus configured to blend the fibers 310. In some embodiments, more than one fiber type is added and blended. A range of fibers may be used to achieve the desired mechanical properties and end-product behavior. These fibers can be combinations of traditional fibers with bi-component fibers that contain a core/sheath design such as PE/PET and/or fibers that contain a bonding agent added during fiber manufacture. In some embodiments, the fibers comprise an additive or inclusion that is added to increase resilience, strength, or other properties of the fiber and thus the substrate and pellet formed thereafter.

In some embodiments, a chute feeder feeds the blended fibers into a carding apparatus 330, where the fiber is carded, block 220. Carding, similarly to the prior art process, is the individualization of fibers for untangling and alignment. After carding, the fiber is a low basis weight material predominantly oriented parallel to the Y axis in the XY plane (parallel to the direction of material flow).

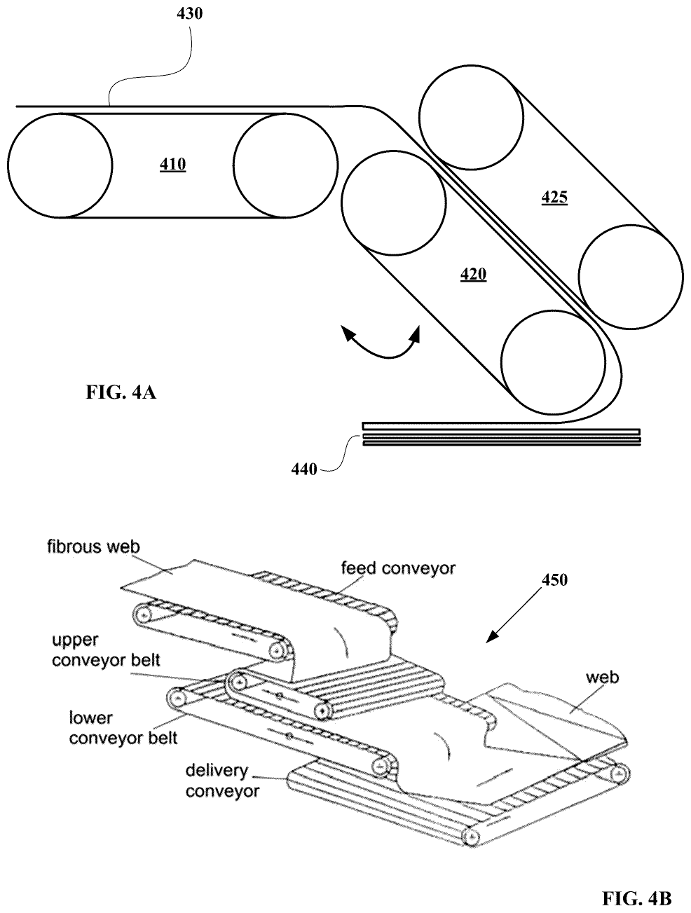

After the fibers are separated and aligned, the process continues to block 230, where the fibers will be cross-lapped in a cross-lapping device 340. Cross-lapping includes layering fibers at a ninety-degree angle. This step is where the weight of the substrate is controlled. The more layers of fiber the higher the weight of the substrate. An exemplary device used for cross-lapping is illustrated in FIG. 4A. A web of fibers 430 that were previously untangled and aligned in the carding process are fed onto a feed conveyer 410. The feed conveyer 410 feeds the web into a series of lap conveyers 420 and 425 to layer or stack several webs to form a substrate 440. Another exemplary cross-lapping device 450 is illustrated in FIG. 4B. In some embodiments, the substrate may be multi-layered, thus producing a multi-layered pellet. This may be achieved by cross-lapping webs comprising different fibers to create multiple layers. In some embodiments, the layers may have varying fiber types.

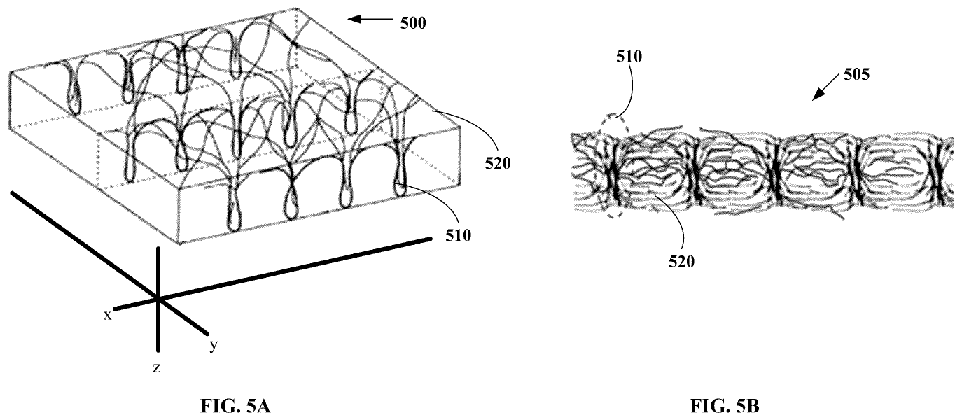

After cross-lapping to form a substrate, the substrate is bonded at block 240. Substrate bonding is performed to bond the fibrous web layers of the substrate together by entangling the fibers perpendicular relative to the plane of the substrate, as illustrated in FIG. 5. FIG. 5A illustrates a perspective view of a bonded substrate 500 and a cross-section view 505. The bonded regions 510 are regions where the fibers are re-oriented along the Z-axis, or perpendicular to the plane of the fibrous web substrate. The non-bonded regions 520 are regions that are not re-oriented and the fibers are still generally parallel with the plane of the fibrous web substrate, or in the XY plane.

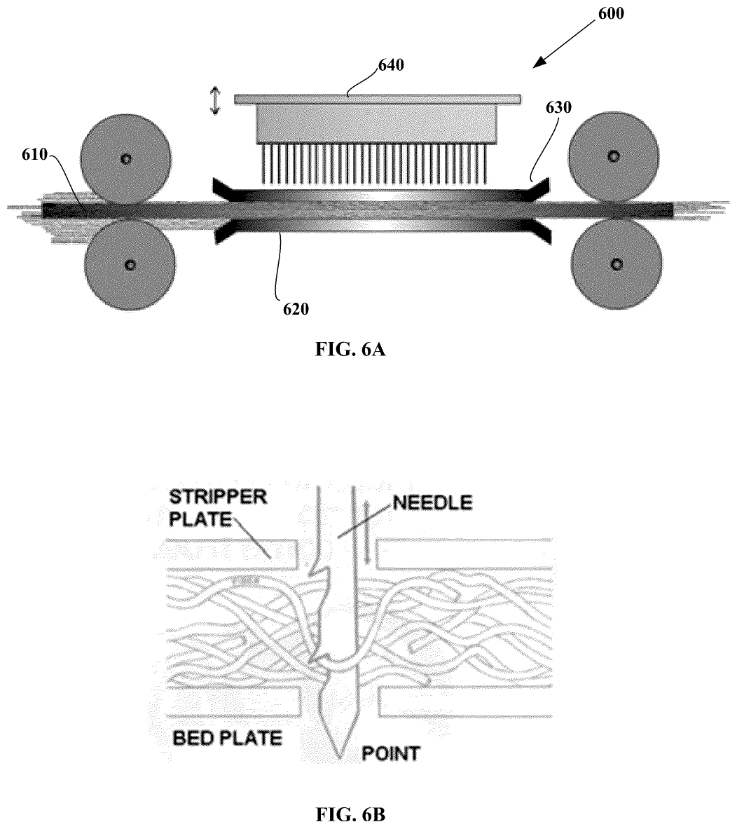

In some embodiments, the process includes needle-punching to bond the substrate, using one or more needle-looms 360 as illustrated in FIG. 3. Needle-punching is illustrated in FIG. 6. FIG. 6A illustrates an exemplary needle-loom 600, wherein web substrate 610 fed between a stripper plate 630 and a bed plate 620 is punched with needles on a needle-board 640. As illustrated in FIG. 6B, the needle-punching process uses a series of barbed needles, that, when inserted into the structure, attach to fibers, and re-orient and entangle fibers in the Z direction. The zones where needles are inserted, the bonding regions, now become points of mechanical strength.

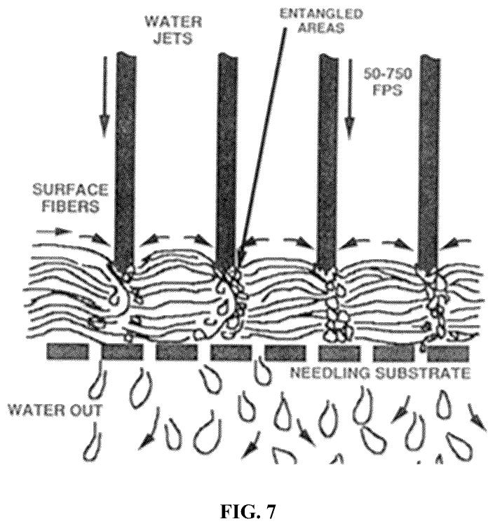

Additionally, or alternatively, hydro-entanglement may be used to bond the substrate. Hydro-entanglement is illustrated in FIG. 7. Hydro-entanglement is the use of high pressure water jets to re-orient the fibers in the bonding region from the XY plane to the YZ and/or XZ planes entangling, or bonding, the fibers. The hydro-entangling process uses high velocity water jets to entangle fibers in the Z direction. Similar to needle-punching, hydro-entangling creates zones of Z direction orientation and entanglement which imparts a change in overall mechanical properties of the structure.

In other embodiments, thermal bonding is used to bond the substrate; examples of thermal bonding include through-air bonding wherein high temperature air is used to slightly melt the exterior surfaces of fibers enabling bonding of the fibers upon cooling. In some embodiments, the substrate is bonded using high temperature and/or pressure. Additionally, or alternatively, ultrasonic bonding is used to bond the substrate. Similar to through-air bonding, using heated and pressurized bonding rolls, or ultrasonic bonding, creates bonds between fibers through fiber melting and attachment upon cooling. Both processes allow for discrete and targeted bonding of substrates that allow for mechanical property manipulation.

The substrate bonding method may be tailored to provide a final product with desired properties for a specific application. For example, through-air bonding creates a more permanent bond than needle-punching or hydro-entanglement, and can enable the creation of a more resilient structure to resist creep and deformation. The method of substrate bonding may also be dependent on the type of fibers present in the substrate.

In some embodiments, after bonding the substrate the bonded substrate is subjected to slitting and winding at block 250 using a winder apparatus 370. Wound substrate may then be stored for pellet-forming 200b at a later time and/or a different location. The process of pellet-forming 200b, begins by unwinding the bound substrate. The unwound substrate is then subjected to die-cutting, at block 270 to produce pellets. In some embodiments, the process may include direct coupling of pellet die-cutting 270 after substrate bonding, removing the steps of slitting and winding 250 and unwinding the substrate 260.

The macro shape of the structure is achieved through the die-cutting process. Die-cutting may be done by rotary or linear (stamp) die-cutting. In both cases, a die is machined with the desired shapes. The shapes can be spaced separately across/around the die surface or adjacent to one another. In some cases, the shapes can be completely connected as to reduce the amount of unused substrate post cutting. Shapes can all be the same or any number of different shapes. For example, a die can be machined to contain all circular shapes, or a die can be machined to contain circles, squares, and diamonds.

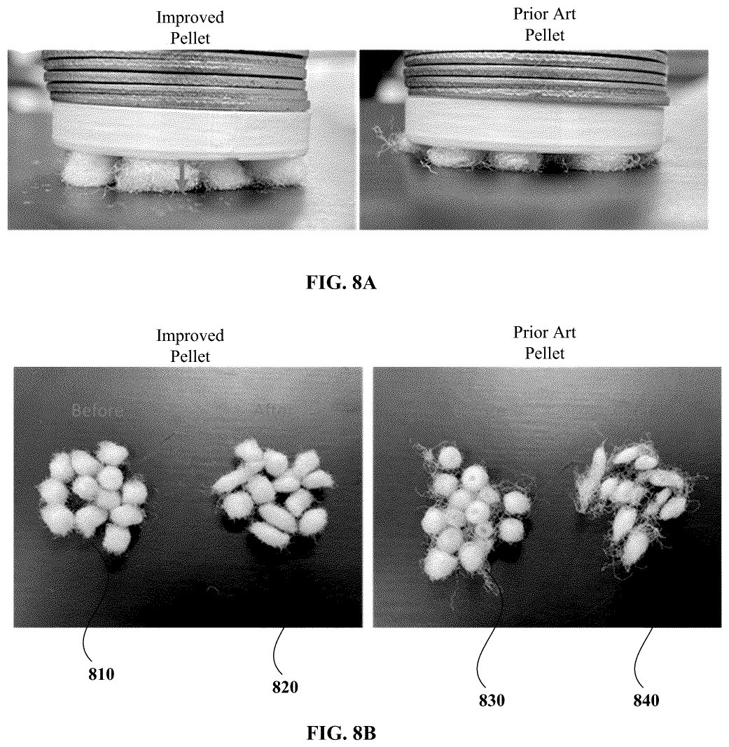

FIG. 8 illustrates the difference in mechanical properties between the prior art pellet and the improved pellet described herein. FIG. 8A illustrates the difference in compressive properties between the two pellets. The improved pellet, described herein, is more resistant to compression than the prior art pellet as is evidenced in FIG. 8A by the application of equivalent compressive force. Additionally, FIG. 8B illustrates the appearance of each pellet both before and after the application of shear force. The improved pellets before the application of shear force 810 have a relatively uniform shape and are intact. After the application of shear force 820, the pellets are slightly less uniform but still remain intact. The prior art pellet before the application of shear force 830 are less uniform in shape and have many extraneous fibers. After the application of shear force 840, the prior art pellets are not as intact and many of the pellets are nearly completely shredded and no longer in pellet-form.

The improved pellet and the prior art pellet are compared in FIG. 9. The top-view of the improved pellet 910 and side view of the improved pellet 900 are shown in FIG. 9. The bonding regions 970 can be seen in the top view of the improved pellet 900. As can be seen in the perspective view of the improved pellet 910, the fibers in the pellet are oriented in the XY plane throughout the pellet, and at the bonding regions 970, the fibers are re-oriented perpendicular to the XY plane. As seen in the top-view of the prior art pellet 930 there is a center void 935. The center void 935 is due to the rolling or spinning process of forming the prior art pellets, where the fibers are all oriented radially about an axis, as can be seen from the perspective view of the prior art pellet 920, forming this void 935 in the center of the pellet. The center void 935 acts as a “dead-zone” for any type of fluid handling or structural needs. In addition to the center void 935, the prior art pellet has an excess of extraneous fibers 925.

Fluid movement through the structure can be defined as both fluid being absorbed into a dry structure and/or fluid being desorbed from a structure. In both cases, pressure is the driving force for fluid movement. By having a XYZ oriented design, movement of fluid throughout the structure will occur according to the weighted fiber orientation. For example, a structure with an equal orientation in the XYZ will have an equal distribution of fluid throughout. If the orientation is dominant in the X axis, fluid will have dominant flow in that direction. This occurs for both fluid entering the structure as well as fluid exiting the structure. In a radially oriented design, as seen in the prior art pellet, fluid will tend to follow paths in the radial direction. This has a negative impact both on the rate of fluid movement as well as full utilization of the total structure for fluid management. Fibers aligned in the Z direction enable fluid movement between layers of the XY plane fibers. This fluid behavior is not possible with the prior art pellet as there is no fiber directionality in the axial plane.

The governance of fluid movement in the pellet can be explained by Jurin's Law, Equation 1. Jurin's law is derived from the Young Laplace equation which governs capillary action of fluid, where h is height, γ is surface tension, θ is contact angle, ρ is fluid density, g is gravity, and r is radius of the capillary.

$$h = \frac{( 2 γ cos q )}{ρ gr} \qquad Equation 1$$

For the improved pellet, described herein, the height can be any distance across the structure, for example, across the X axis, the Y axis, or the Z axis. Surface tension, contact angle, and fluid density can be considered constants, with only the radius changing as fiber orientation changes. Accordingly, as radius decreases, the distance that fluid can travel (h) increases. In some embodiments, the improved pellets may have a distribution of radii which will therefore have a distribution of fluid travel distances. Additionally, the improved pellets, as described herein, may have zones of pore sizes and directionality of pores which enable fluid movement scenarios not possible with the prior art pellet.

The properties, mechanical and fluid dynamic properties, of the fibrous pellet may be tailored through a variety of processing conditions. One such way to tailor the properties is to change the orientation of the fibers in the XY, YZ, and XZ planes. To promote fiber orientation changes, cross-lapping in combination with hydro-entangling and/or needle-punching are utilized. Cross-lapping can be tailored to adjust substrate basis weight, fiber orientation in the X and Y planes. The density of the fibers may also be used to tailor the properties of the pellet.

In some embodiments, the fibrous pellet may be used as a smoke-less and tobacco-free nicotine pouch as described in U.S. patent application Ser. No. 17/178,108, which is hereby incorporated by reference in its entirety.

In some embodiments, the fibrous pellet may be configured to provide a fibrous pellet with time-delayed release of active ingredients. This may be achieved with a pellet with heterogeneous porosity. In some embodiments, a fibrous pellet with heterogeneous porosity may be prepared by incorporating hollow fibers with solid fibers. Additionally, or alternatively, heterogeneous porosity may be achieved by incorporating differently shaped fibers. In other embodiments, the macro-shape of the pellet may provide a pellet with heterogeneous porosity.

In some embodiments, the fibrous pellet may be configured to have a dynamic heterogeneous morphology. A pellet with dynamic heterogeneous morphology may be obtained by tailoring the mechanical properties of the pellet (by altering fiber direction, density, etc as described previously) or by using a fiber with desired properties, such as glass transition temperature. The pellet shape changes due to forces exerted during application or by the tongue, cheek and gum when in the mouth of a user. A pellet with a dynamic heterogeneous morphology enables a fitted pellet for comfort and/or proper positioning of the pellet.

In some embodiments, the fibrous pellet may be configured to have dynamic heterogeneous porosity and permeability. A pellet with dynamic heterogeneous porosity and permeability may be obtained by tailoring the mechanical properties of the pellet, as previously described. Along with pellet shape change, pellet compression and shear change occurs with pellet adjustment in the oral cavity. This creates a shift in the pore size distribution that is unique to each pellet depending upon the pellet's orientation relative to the forces applied, giving a discontinuous porosity and permeability profile which enables varying active release behavior during use.

In some embodiments, the pellet comprises an active ingredient. Active ingredients include, but are not limited to, nicotine, nitroglycerin, cannabinoid and cannabinoid derivatives, and any other active ingredient capable of absorption by the oral mucosa. In some embodiments, the active ingredient may be applied and absorbed into the pellet after pellet-forming. In some embodiments, the active ingredient is applied and absorbed into the substrate after cross-lapping. Additionally, or alternatively, the active ingredient may be applied and absorbed into the substrate after bonding. In some embodiments, the active ingredient is applied to the fiber after carding. Additionally, or alternatively, the active ingredient is applied to the fiber prior to carding. The active ingredient may be applied in a solution, or carrier liquid, with other additives such as flavorings.

### WORKING EXAMPLES

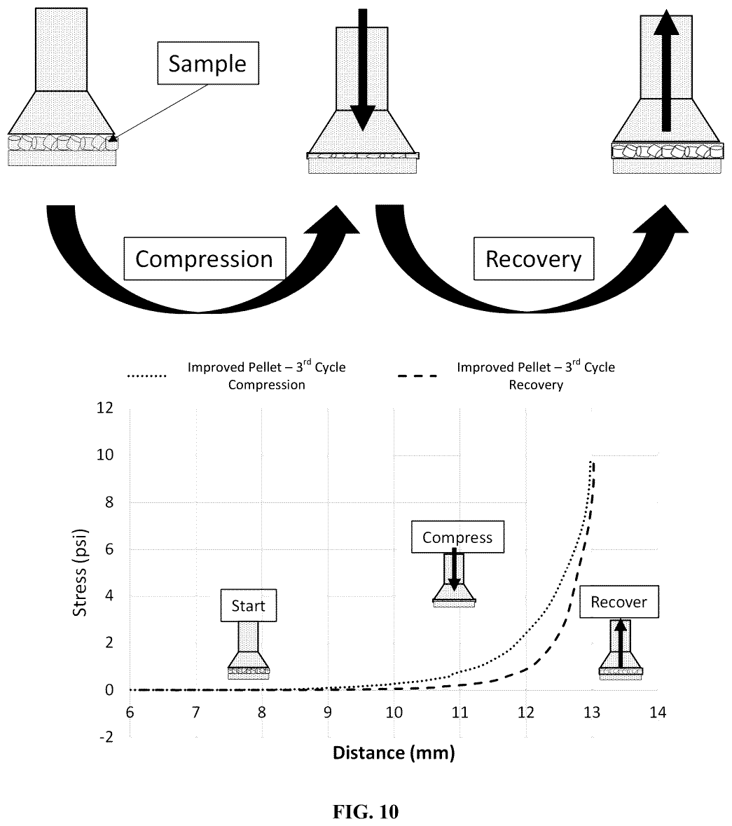

To test the recovery of the pellet after compression, the compression test illustrated in FIG. 10 is conducted. A fourth of a gram of pellets are placed into a container with a height of 10 mm and an inner diameter of 39 mm. The container is placed on the lower platform of a compression tester that is set to a speed of 1 mm/s. The pellets are compressed using a compression foot with a diameter of 38 mm and a holding step of 10 seconds at 100% compression. This is repeated for 3 cycles, and the force and distance for each cycle are recorded. A chart in FIG. 10 shows the stress vs distance curves of the pellets of an embodiment of the present invention being compressed and recovering during the 3^{rd} cycle of the compression test.

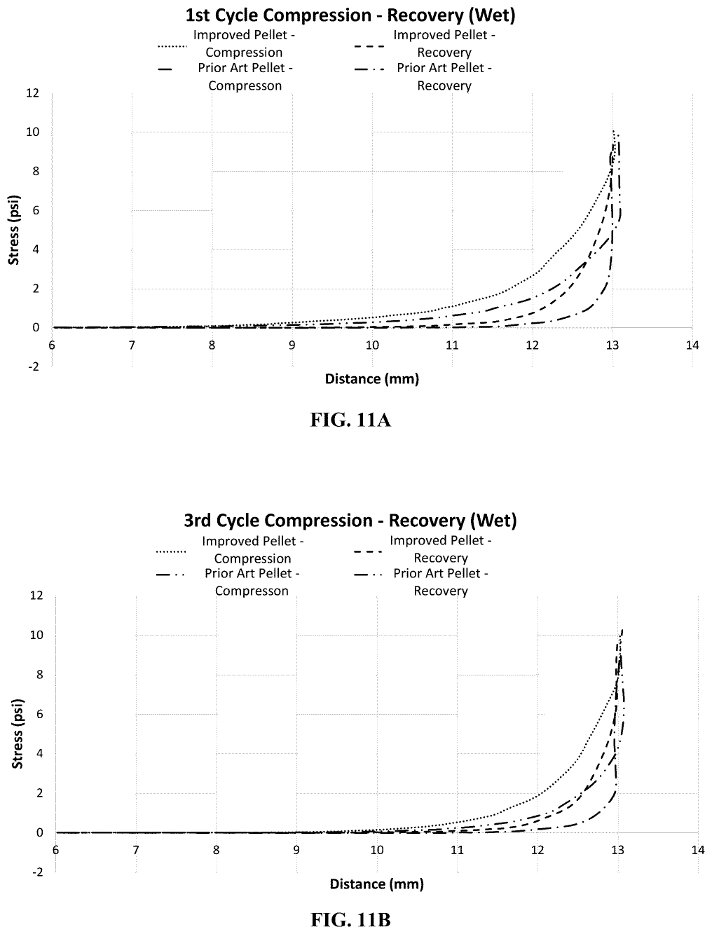

FIGS. 11A and 11B show a stress vs distance curve for compression and recovery of both the prior art and the present invention during different cycles of the compression test while wet. The x-axis refers to the distance the foot of the compression foot travels. FIG. 11A shows the stress exhibited by the pellets during the 1^{st} cycle of the compression test. Both the compression and the recovery curves of the present invention exhibit more stress than those of the prior art. This means that the present invention is more prone to maintain its original shape when wet than the prior art. A similar trend is seen in FIG. 11B during the 3^{rd} cycle of the compression test. The prior art curves for compression and recovery show a lack of pushback onto the compression foot when wet. In oral applications, this translates to the prior art losing shape when it becomes wet by saliva and compressed by the mouth and gums.

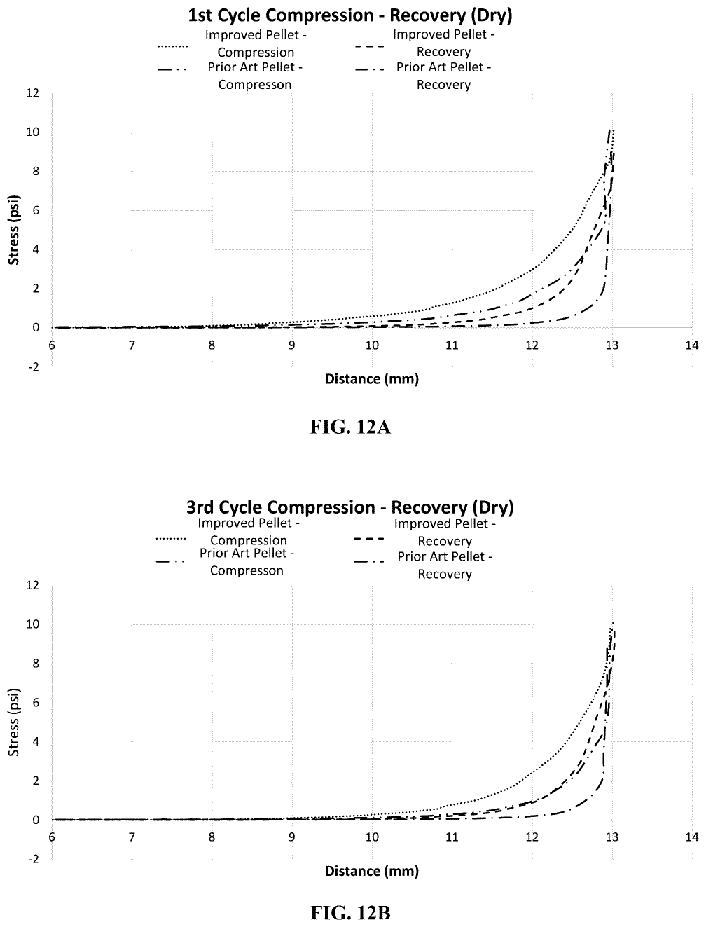

FIGS. 12A and 12B show a stress vs distance curve for compression and recovery of both the prior art and the present invention during different cycles of the compression test. This compression test is of the method described in FIG. 10. The near vertical slope of the recovery curve of the prior art in both FIGS. 12A and 12B after being compressed fully indicates that the pellet is not pushing back on the foot of the device and thus not retaining its original shape.

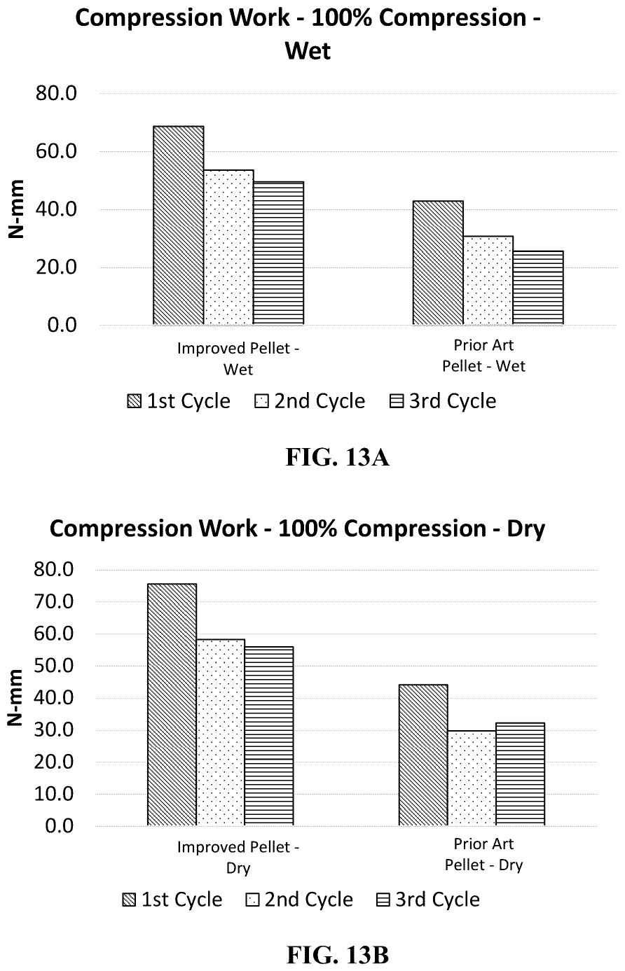

FIGS. 13A and 13B show the work in N-mm that both wet and dry pellets exert on the compression foot while at 100% compression. In both FIGS. 13A and 13B, the work done on the compression foot during the 3^{rd} cycle of compressing the present invention is greater than the work done during the 1^{st} cycle of the prior art. This shows that regardless of if the pellet is wet or dry, the present invention is likely to push back on the compression foot with more force than the prior art. This shows the present invention's quality and tendency to retain its original shape.

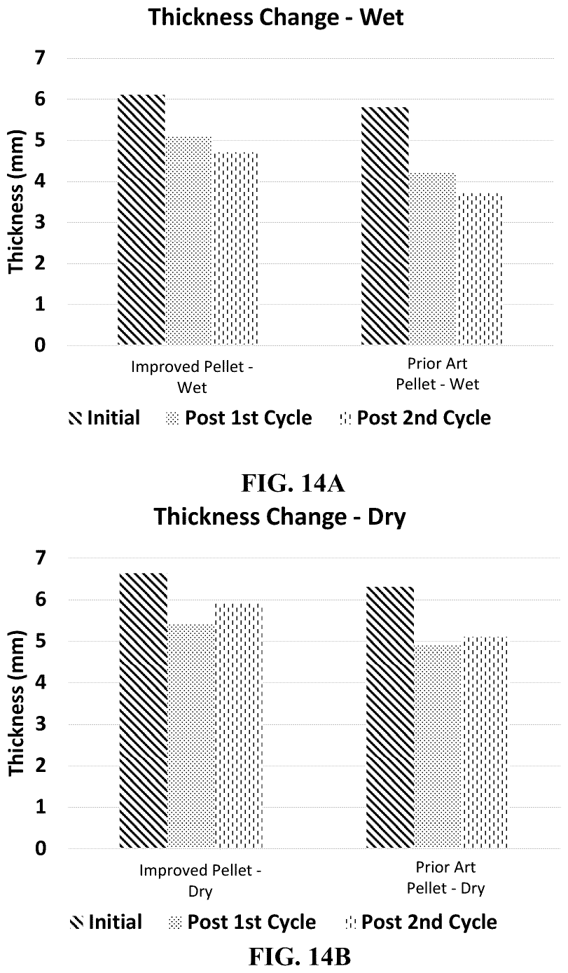

FIGS. 14A and 14B show the change in thickness after a two-cycle compression test, with the thickness being measured initially (prior to compression), after the first cycle, and after the second cycle. This test compares wet and dry thickness changes between the present invention and the prior art. The improved fibrous pellet of the present invention shows less thickness change after compression compared to the prior art pellet.

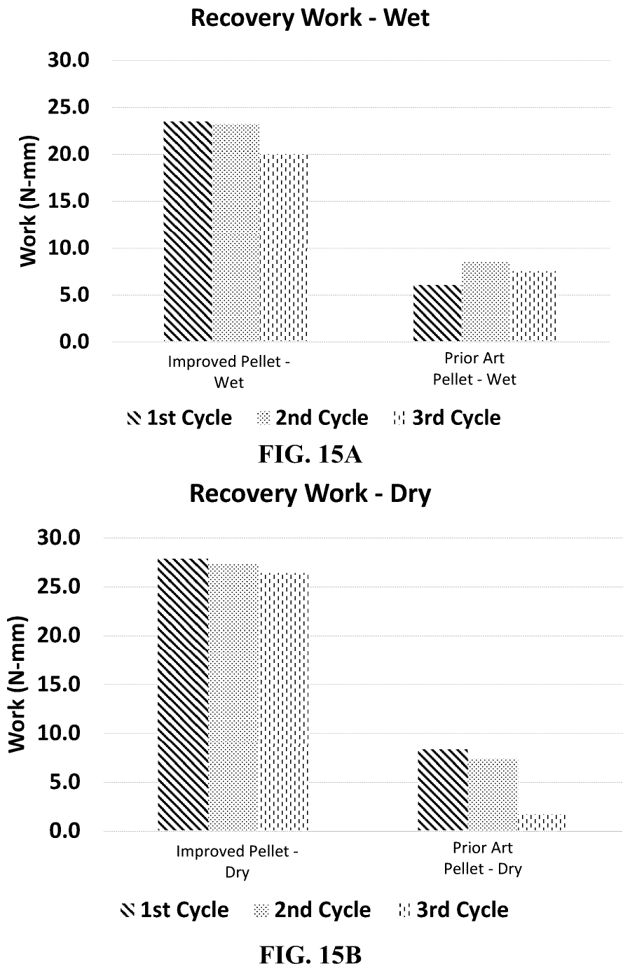

FIGS. 15A and 15B show the recovery work post-compression test of the pellet while both wet and dry. Compared to the prior art, the improved pellet of the present invention shows approximately four-fold more work, illustrating that the improved pellet of the present invention has more resiliency than the prior art pellet.

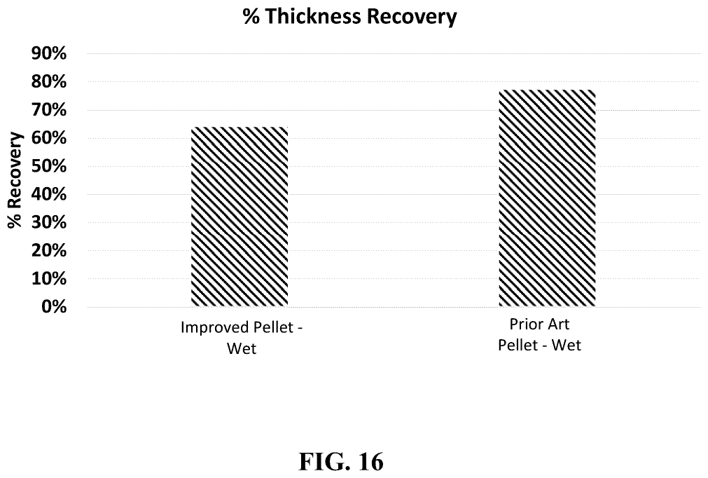

FIG. 16 shows the percent thickness recovery of the improved pellet and the prior art when wet. The improved pellet of the present invention shows a better percent thickness recovery over the prior art pellet.

The fluid retention of the pellet was measured using two different tests: the squeeze out test and the free fluid lost test. The squeeze out test measures the amount of fluid lost by squeezing, or compressing, the pellet. The pellet is put on top of filter paper and squeezed, and the filter paper is measured for the change in weight. The free fluid lost test measures the amount of fluid lost after the pellet sits on a piece of filter paper for one minute by using the same process of weighing the filter paper before and after the test.

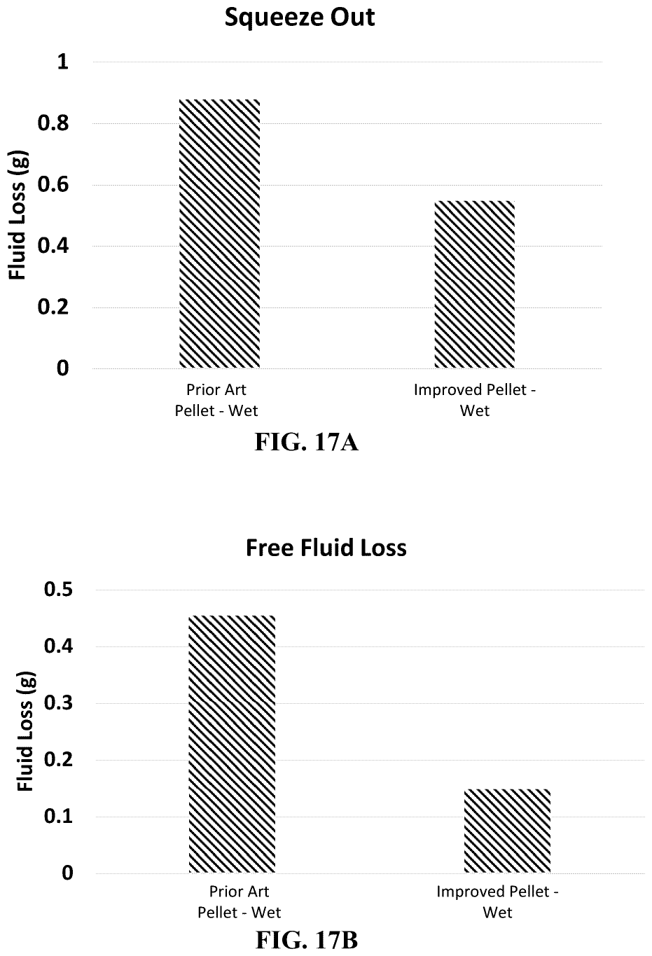

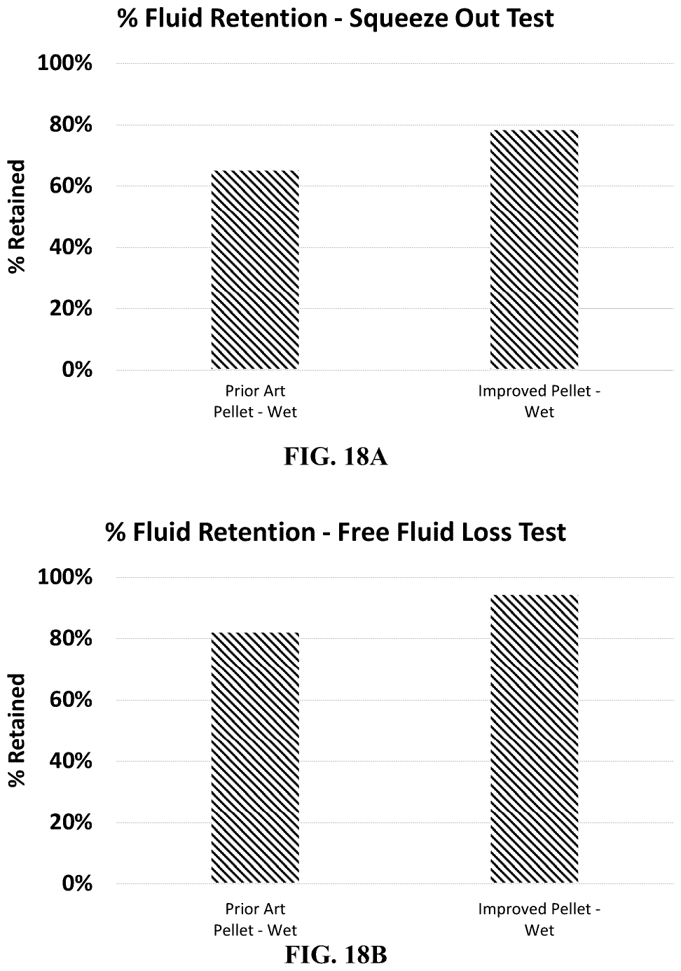

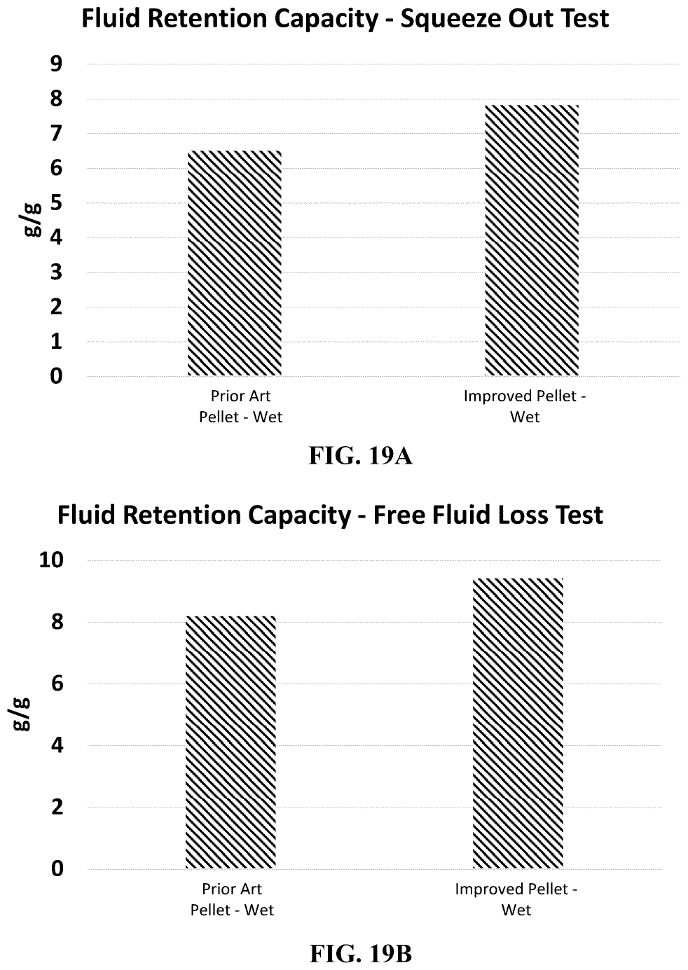

The fluid lost measured during a squeeze out test is shown in FIG. 17A, comparing the improved pellet to the prior art pellet. FIG. 17B shows the fluid lost in a free fluid loss test. FIGS. 18A and 18B show the amount of fluid retention (measured in percent fluid retained) during these tests, comparing the prior art and the present invention. FIGS. 19A and 19B illustrate the amount of fluid retention (measured in gram[s] of fluid per gram of pellet) during these tests, comparing the prior art and the present invention. The improved pellet is shown to have improved fluid retention capacities when compared to the prior art. The improved pellet shows less fluid lost in both the squeeze out test and the free fluid lost test illustrating that the improved pellets retain fluids better than the prior art pellet.

The foregoing description provides embodiments by way of example only. It is envisioned that other embodiments may perform similar functions and/or achieve similar results. Any and all such equivalent embodiments and examples are within the scope of the present invention.

What is claimed is:

1. A fibrous pellet comprising a plurality of fibers generally oriented linearly relative to the X-axis, the Y-axis, and the Z-axis of the pellet.

2. The fibrous pellet according to claim 1, wherein the plurality of fibers comprise an oil based synthetic fiber.

3. The fibrous pellet according to claim 1, wherein the plurality of fibers comprise a cellulosic based fiber.

4. The fibrous pellet according to claim 1, wherein the plurality of fibers comprise a combination of an oil based synthetic fiber and a cellulosic based fiber.

5. The fibrous pellet according to claim 1, wherein the plurality of fibers comprise polyester.

6. The fibrous pellet according to claim 1, wherein the plurality of fibers comprise a bi-component fiber.

7. The fibrous pellet according to claim 1, wherein the plurality of fibers comprise a combination of polyester, polypropylene, and bicomponent fibers.

8. The fibrous pellet according to claim 1, wherein the plurality of fibers comprise a combination of a cotton, polyester and bicomponent fibers.

9. The fibrous pellet according to claim 1, wherein the pellet has a dynamic heterogeneous morphology.

10. The fibrous pellet according to claim 1, wherein the fibrous pellet further comprises an active ingredient.

11. A method of manufacturing a fibrous pellet, the method comprising:
carding a fiber to generate aligned fibers,
cross-lapping the aligned fibers to create a layered substrate,
bonding the layered substrate to create bonded substrate, and
die-cutting the bonded substrate into pellets.

12. The method of manufacturing a fibrous pellet according to claim 11, wherein the method further comprises applying an active ingredient to the fiber, the aligned fiber, the layered substrate, the bonded substrate, or the pellets.

13. The method of manufacturing a fibrous pellet according to claim 11, wherein bonding the layered substrate comprises hydro-entangling, needling-punching, thermal bonding, ultrasonic bonding, or combinations thereof.

14. A fibrous pellet formed by the process of carding a fiber to generate aligned fibers,
cross-lapping the aligned fibers to create a layered substrate,
bonding the layered substrate to create bonded substrate, and
die-cutting the bonded substrate into pellets.

15. The fibrous pellet according to claim 14, wherein the process further comprises applying an active ingredient to the fiber, the aligned fiber, the layered substrate, the bonded substrate, or the pellets.

16. The fibrous pellet according to claim 14, wherein bonding the layered substrate comprises hydro-entangling, needling-punching, thermal bonding, ultrasonic bonding, or combinations thereof.

17. The fibrous pellet according to claim 14, wherein the fiber comprises a cellulosic based fiber.

18. The fibrous pellet according to claim 14, comprising a plurality of fibers generally oriented linearly relative to the X-axis, the Y-axis, and the Z-axis of the pellet.

19. The fibrous pellet according to claim 14, wherein the fiber comprises polyester.

20. The fibrous pellet according to claim 14, wherein the fibrous pellet further comprises an active ingredient.
